(19) United States
(12) Patent Application Publication
(10) Pub. No.: US 2011/0230759 A1
(43) Pub. Date: Sep. 22, 2011

(54) MEDICAL IMAGING DEVICE COMPRISING RADIOGRAPHIC ACQUISITION MEANS AND GUIDE MEANS FOR ULTRASOUND PROBE

(76) Inventor: Serge Louis Wilfrid Muller, Buc (FR)

(21) Appl. No.: 13/050,741

(22) Filed: Mar. 17, 2011

(30) Foreign Application Priority Data
Mar. 17, 2010 (FR) 1051897

(51) Int. Cl. A61B 6/00; A61B 8/00

(52) U.S. Cl. 600/427

(57) ABSTRACT

A device for medical imaging is provided. The device includes an X-ray acquisition means configured to acquire data representative of an object. The X-ray acquisition means includes; an arm comprising a radiation source; a radiation detector; a planar object support; and a pad. The device further includes an ultrasound probe capable of being shifted manually by a user; a processor configured to produce at least one image of the object from data acquired by the X-ray acquisition means; and determine a path between a position of the ultrasound probe and a position in the object corresponding to a selected zone in said image; and a guide of the ultrasound probe configured to facilitate shifting the ultrasound probe along the path between a position of the ultrasound probe and the position in the object corresponding to the selected zone in the at least one image.

### FIELD OF INVENTION

The field of the invention relates generally to medical imaging, and more particularly to a medical imaging device for mammography.

### BACKGROUND OF THE INVENTION

Radiography and echography are two imaging techniques used for exploration of the breast.

Radiography produces an image of internal structures of an object from X-rays passing through these internal structures.

Echography produces an image of the internal structures of an object from ultrasound reflected by said internal structures.

It is known today that radiography and echography are two complementary techniques used for examining the breast.

Medical imaging devices combining these two imaging techniques have therefore already been proposed.

For example, the document U.S. Pat. No. 5,938,613 describes an apparatus combining X-ray mammography equipment with an ultrasound transducer. This apparatus generates images of the internal structure of breast tissue from X-ray and ultrasound. The transducer is mounted on a sled which moves by means of a motor and is carried out in increments. The sled moves on a transparent X-ray and ultrasound compression plate of the breast. This apparatus has drawbacks. Shifting the sled in increments limits possibilities for generating images from the transducer. The sled on which the transducer is mounted moves on the compression plate and takes images only according to the direction perpendicular to the compression plate. The user cannot move the transducer as rapidly and freely as preferred and cannot conduct conventional examination via ultrasound. If the user wants to take images via ultrasound according to a direction transverse to this direction, the patient must change position.

Imaging apparatus comprising an X-ray mammography system and an ultrasound probe capable of being moved manually by a user has also been proposed in the document FR 2,835,731. This apparatus comprises a positioning probe of the ultrasound probe. This imaging apparatus more easily matches mammographic images and echographic images. However, it is difficult for the user to obtain mammographic and echographic images of the same zone of the breast using the apparatus described in FR 2,835,731.

In one embodiment, a device for taking diverse images of a given zone of the breast by X-ray and by ultrasound using the same acquisition geometry for acquisition via X-ray and ultrasound is disclosed.

### SUMMARY OF THE INVENTION

For this purpose, an imaging device is provided comprising: X-ray acquisition means configured to acquire data representative of an object. The X-ray acquisition means comprising: an arm comprising a radiation source for emitting X-rays; a radiation detector configured to receive the X-rays; a planar object support placed between the radiation source and the radiation detector; and a pad placed between the planar object support and the radiation source. The medical imaging device further includes an ultrasound probe capable of being shifted manually by a user and a processor configured to produce at least one image of the object from data acquired by the X-ray acquisition means, and determine a path between a position of the ultrasound probe and a position in the object corresponding to a selected zone in said image. The medical imaging device further comprises a guide of the ultrasound probe configured to facilitate shifting the ultrasound probe along the path between a position of the ultrasound probe and the position in the object corresponding to the selected zone in the at least one image.

### BRIEF DESCRIPTION OF THE DRAWINGS

Other characteristics and advantages of the device and of the method according to embodiments of the invention will emerge from the following description which is purely illustrative and must be considered in conjunction with the attached diagrams, in which:

FIG. 1 is a schematic representation of a medical imaging device according to one embodiment of the present invention.

FIG. 2 is a schematic representation of a medical imaging method according to one embodiment of the present invention.

FIGS. 3a and 3b are schematic representations of a medical imaging device according to one embodiment of the present invention.

### DETAILED DESCRIPTION OF THE INVENTION

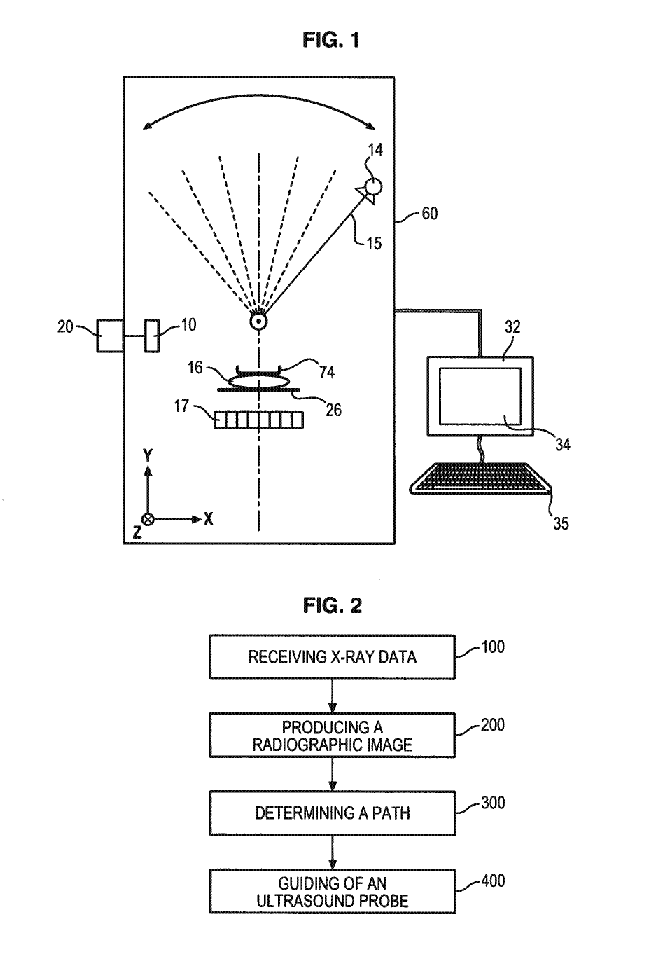

Embodiments of the invention will now be described in greater detail in reference to FIGS. 1, 2, 3a and 3b. In the following description, the object 16 to be imaged is a breast of a patient. However, it is understood that embodiments of the present invention can be applied to imaging of other objects.

FIG. 1 represents a medical imaging device comprising X-ray acquisition means, an ultrasound probe 10, a processor 32 and a guide 20.

The X-ray acquisition means comprise an arm 15 containing a radiation source 14 for emitting X-rays, a radiation detector 17 for receiving the X-rays, a planar object support 26 placed between the source 14 and the detector 17, a pad 74 placed between the object support 26 and the source 14 for compression of the object 16 to be imaged. The compression pad 74 is preferably transparent to X-ray and ultrasound. The arm 15 bearing the source 14 can be moved into a plurality of positions 15. This arm 15 plays the role of positioner. As for the source 14, it is mounted pivoting on the arm 15 to allow orientation of the latter relative to the object support 26.

The ultrasound probe 10 enables emission and receiving of ultrasound. The ultrasound probe 10 is capable of being shifted manually by a user. A 2D probe 10 for acquiring a set of 2D data on the breast 16 in the form of cups can be used. Interpolation of this 2D data set generates 3D reconstruction of the structure of the breast tissue. A 3D (or even 4D) probe 10 for acquiring a data set having 3D reconstruction of the structure of the breast tissue can also be used.

The processor 32 is for receiving data sent by the X-ray acquisition means on the one hand. The processor 32 is able to produce a plurality of radiographic projections each corresponding to a respective position of the radiation source from the data received from the acquisition means. The processor 32 is also able to utilize reconstruction processes for obtaining 3D information from radiographic projections. The processor 32 is also able to receive the data emitted by the ultrasound probe. The processor 32 is adapted to produce one (or more) echographic image(s) from these data.

The guide 20 guides the shifting of the ultrasound probe 10, as will be explained in greater detail hereinbelow. The guide 20 is for example a haptic arm at the end of which the ultrasound probe is fixed. The haptic arm preferably has a power return. It should be remembered the term “haptic” designates any technology for touching and manipulating virtual and/or distant objects. Use of a haptic arm “slides” the ultrasound probe 10 on the surface of a virtual volume produced by the processor. The haptic arm can be integrated in or separate from the X-ray acquisition means. In all cases, the position of the haptic arm is known. A calibrating procedure determines the mathematical transformation for moving from the reference of the acquisition means to the reference of the haptic arm.

When a user detects a region of interest in a mammography, he may want to obtain extra information on this region by using an ultrasound probe to make an echographic image of the region of interest. Actually, the echographic image is likely to provide the user with extra information helping him confirm diagnosis.

Yet, with the devices of the prior art, locating a region in the breast is difficult. In fact, the user has only the mammographic image for finding the suspect region to then produce the echographic image. This causes a significant increase in the length of examination.

Due to the time necessary for finding the suspect region to then produce the echographic image, it is frequently necessary to remove the pad, with compression exerted by the latter being a source of discomfort for the patient. This causes a variation in the geometric configuration of the breast which can cause errors in matching the echographic and mammographic images.

Embodiments of the present invention provide a solution for orienting the user in positioning the ultrasound probe 10 on a breast 16 which remains compressed throughout examination. In this way, the geometric configuration of the breast is identical during acquisition of the radiological and echographic images, which lowers the risk of error during matching of these images.

In fact, the presence of guide 20 in the imaging device allows the user to more quickly find a suspect region identified in the mammographic image, and therefore more quickly and easily take images using the ultrasound probe. It is no longer necessary to remove the pad, allowing mammographic and echographic images to be arranged in which geometric configuration of the breast is identical.

An operating principle is the following; once the object to be imaged is positioned on the object support and compressed by the pad, data representative of the object are acquired using the X-ray acquisition means.

The processor receives 100 these data, and produce 200 radiographic images of the object (in 2D or 3D) from the acquired data.

The radiographic images are used for selecting one (or more) region(s) of interest in the breast. This locating can be done manually by the user using gripping means of the device. This locating can also be done automatically by the processor using a radiological image—analysis process known to the person skilled in the art—for example CAD software (acronym for “Computer Aided Detection”). The region of interest can be a point, trimming or a predefined form.

Knowing the position of the region of interest in the radiographic images, the processor 32 determines 300 a path 30 between the position of the ultrasound probe and a position in the breast corresponding to the selected region of interest. More precisely, from the position of the region of interest in the radiographic images, the processor 32 determines the position of this region in the breast (the geometric configuration of the breast and the geometry of the acquisition means being known). Also, since the geometry of the haptic arm is known, the position of the probe which is fixed at its end is also known. The processor therefore have sufficient information for determining a path between the position of the ultrasound source and the position in the breast corresponding to the selected region of interest.

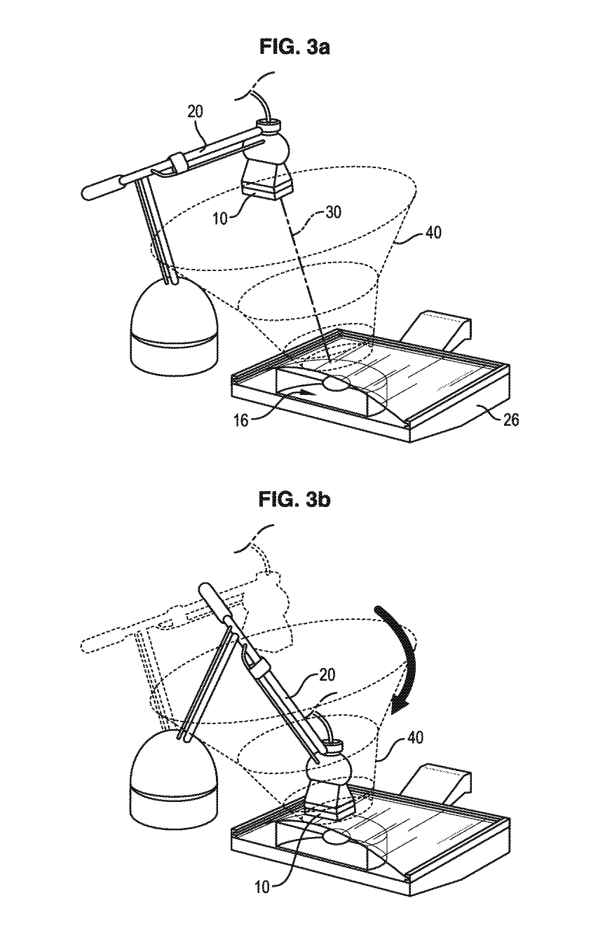

Advantageously, the processor 32 can determine a virtual volume 40 in which the path is registered. This virtual volume 40 guides the movement of the user to move the probe to the breast of the patient, as illustrated in FIGS. 3a and 3b. This virtual volume can have different forms. The form of the virtual volume can be for example a truncated cone, or a truncated torus. The virtual volume preferably has a funnel shape so as to progressively channel shifting of the probe to the position of the region of interest in the breast, but also to orient the ultrasound probe according to an axis parallel to the X-ray beam emitted by the acquisition means.

In this way, embodiments of the present invention utilize radiographic images to construct a virtual volume which limits shifting of the ultrasound probe connected to a haptic device such that the user is guided 400 as far as the position on the breast where the ultrasound probe will be capable of displaying the selected region of interest on radiographic images. This reduces the duration of the examination.

In a variant embodiment, the processor 32 is able to display the path 30 for each new position of the ultrasound source. This defines a new path and a new virtual volume, especially assuming that the user has shifted the ultrasound probe out of the virtual volume.

The reader will have understood that numerous modifications can be made without departing materially from the new ideas and advantages described here.

For example, in the case where the region of interest is a point, a target region can be determined by the processor. This target region is obtained for example by using region-enhancing processes or any other process known to the person skilled in the art.

Consequently, all modifications of this type are intended to be incorporated within the scope of the system and imaging method such as defined in the attached claims.

What is claimed is:

1. A medical imaging device, comprising:
an X-ray acquisition means configured to acquire data representative of an object, the X-ray acquisition means comprising:
an arm comprising a radiation source for emitting X-rays;
a radiation detector configured to receive the X-rays;
a planar object support placed between the radiation source and the radiation detector; and
a pad placed between the planar object support and the radiation source;
an ultrasound probe capable of being shifted manually by a user;
a processor configured to:
produce at least one image of the object from data acquired by the X-ray acquisition means; and
determine a path between a position of the ultrasound probe and a position in the object corresponding to a selected zone in said image; and
a guide of the ultrasound probe configured to facilitate shifting the ultrasound probe along the path between a position of the ultrasound probe and the position in the object corresponding to the selected zone in the at least one image.

2. The device of claim 1, wherein the guide comprises a positioning probe of the ultrasound probe, said guide configured to send information on the position of the ultrasound probe to the processor.

3. The device of claim 1, wherein the processor is configured to display the path between the position of the ultrasound probe and the position in the object corresponding to the selected zone for each new position of the ultrasound probe.

4. The device of claim 1, wherein the processor is configured to determine a volume in which the path is registered.

5. The device of claim 1, wherein the guide comprises a haptic arm with power return.
